(19) United States
(12) Patent Application Publication
(10) Pub. No.: US 2006/0121001 A1
(43) Pub. Date: Jun. 8, 2006

(54) Method for preparing animal or human adult stem cells and therapeutic use thereof

(75) Inventors: Didier Montarras, Sevres (FR); Nicolas Borenstein, Paris (FR); Christian Pinset, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: Institut Pasteur, Paris cedux 15 (FR); Celogos, Paris (FR); Fondation De L'avenir, Paris (FR); Centre National De La Recherche Scientifique, Paris (FR)

(21) Appl. No.: 10/517,774

(22) PCT Filed: Jun. 27, 2003
(86) PCT No.: PCT/FR03/02010

§ 371 Date: Oct. 13, 2005

(30) Foreign Application Priority Data
Jun. 28, 2002 (CA) 2391638

(51) Int. Cl. A61K 35/12; C12N 5/08

(52) U.S. Cl. 424/93.7; 435/325; 435/366

(57) ABSTRACT

The invention relates to tissue regeneration and repair by cell transplant. More particularly, the invention concerns a method for preparing adult stem cells which preserves the diversity and plasticity of said cells. The invention also concerns the use of said stem cells obtained by the inventive extraction method for therapeutic uses, including gene and cell therapy. The inventive preparation method essentially consists in: cell extraction, mechanical dissociation, enzymatic dissociation, maintaining the cells obtained in a specific culture medium enabling preservation of diversity and plasticity, said method excluding cell culture.

### FIELD OF THE INVENTION

The present invention relates to the field of tissue regeneration and repair by cell transplantation. More particularly, the present invention relates to a method for preparing adult stem cells which preserve the diversity and plasticity of these cells. The present invention also relates to the use of these stem cells, obtained by means of the proposed extraction method, in therapeutic applications, including gene and cell therapy.

### DESCRIPTION OF THE PRIOR ART

For anatomical and biological reasons, the blood and its derivatives were the first cells transplanted. Hematopoietic cell transplants have experienced a similar path. For about twenty years, the transfer of fetal neuronal cells has been studied as a therapeutic approach in neurodegenerative pathologies such as Parkinson's disease and more recently Huntington's chorea. The clinical results are coherent and show a clear improvement. The major limitation of the latter approach is the source of transplanted cells. In fact, the use of fetal neuronal cells makes the spread of this type of therapeutic approaches difficult. Keratinocyte autografts constitute a therapeutic solution in the case of third degree burns. One advantage of this system is the ability of keratinocytes, the main cells of the epidermis, to be cultured ex vivo. Along the same lines, it has been possible to reconstruct defective cartilages using chondrocytes multiplied in culture and then grafted by arthroscopy. Similar approaches, that are still very experimental, are also in the process of being carried out for organs such as the pancreas and the liver.

These data, still too limited, demonstrate the potential of regenerative medicine by cell therapy. There are several types of limitations to the extension of these approaches. In order to be capable of compensating for the failings of a tissue by cell therapy, it is necessary to have cell sources corresponding to the following characteristics:

the cells must be easy to sample;

they must survive at the injection sites;

they must exhibit a good capacity for proliferation and colonization;

they must be capable of differentiating in order to obtain a functional result.

There is therefore a need for new methods for obtaining stem cells capable of being used, inter alia, in the context of tissue regeneration and repair by cell transplantation.

### SUMMARY OF THE INVENTION

The present invention proposes a method of cell preparation that corresponds to the abovementioned need. More particularly, the present invention is directed toward a method of cell preparation capable of preserving the diversity and the plasticity of vertebrate stem cells preferably originating from a tissue biopsy for their subsequent use in the context of tissue regeneration or repair by transplantation in an animal, such as a human being.

The invention is also directed toward a defined culture medium, but is particularly useful during the implementation of the method of cell preparation.

The present invention is also directed toward the cell preparation or the stem cells obtained by means of the preparation method of the invention.

The present invention is also directed toward the use of these stem cells, obtained by means of the proposed preparation method, in therapeutic applications, including gene or cell therapy.

The present invention is also directed toward a cell composition comprising human or animal stem cells obtained according to the preparation method of the invention.

The present invention also relates to a method for preparing stem cells capable of repairing or of regenerating, in vivo, tissues or organs of a vertebrate.

The present invention also relates to a method of treatment, characterized by the implantation of autologous or heterologous animal stem cells obtained according to the method of the invention, in an animal.

The originality of the present invention relates to the fact that, unlike the cell preparation methods used in the field, the preparation method of the present invention makes it possible:

to extract the cells under defined conditions without animal protein of extractive origin, and therefore under conditions that are safe from a health point of view;

to maintain the cell viability by using antioxidant and anti-apoptotic molecules;

to preserve the cell diversity;

to maintain the cellular plasticity potential;

to maintain the differentiation potential;

to improve the implantation, the colonization and the differentiation of the grafted cells; and

to transplant the cells without having to involve an in vitro expansion step in culture.

For example, in the cell preparation method described in the article by Pouzet et al. (2000. Circulation. 102:III2210-5), there is no cell extraction or isolation according to a method of enzymatic digestion. As an alternative, the muscle tissue is chopped up. In addition, there is no cell preparation step as understood within the meaning of the present invention. The conclusion of this study is, moreover, to the effect that it would not be possible to bypass the in vitro expansion phase in culture, the functional recovery as discussed in the article by Pouzet et al. depending rather on the number of cells. According to the method of the invention, the method of preparing the cells advantageously results, without in vitro expansion in culture, in very substantial colonization and also in rapid functional recovery of the treated organ.

### DESCRIPTION OF THE FIGURES

FIGS. 1 to 5 are micrographs illustrating the demonstration of grafted muscle cells in ewe heart. The cells were obtained by enzymatic dissociation of a skeletal muscle biopsy and then directly implanted, without any in vitro expansion phase in culture, into the myocardium of the same ewe (autologous graft). The presence of skeletal muscle cells is revealed using the MY32 antibody which specifically recognizes the heavy chain of skeletal myosin.

FIG. 1 illustrates the detection of grafted skeletal muscle cells with the MY32 antibody by means of the presence, inside the myocardial wall, of massive graft zones (2 to 9 mm in diameter) or of discrete loci (scale bar, 250 microns).

FIG. 2 illustrates the immunodetection of skeletal muscle cells with the MY32 antibody. In the myocardial wall, the fibers labeled with the MY32 antibody are generally aligned with the network of native cardiomyocytes. Some fibers are intensely labeled whereas others are only weakly labeled; the native cardiomyocytes are not labeled (scale bar, 500 microns).

FIG. 3 shows grafted cells differentiating into muscle fibers and forming organized sarcomeres (scale bar, 25 microns).

FIG. 4 illustrates the immunohistochemical specificity of the MY32 antibody.

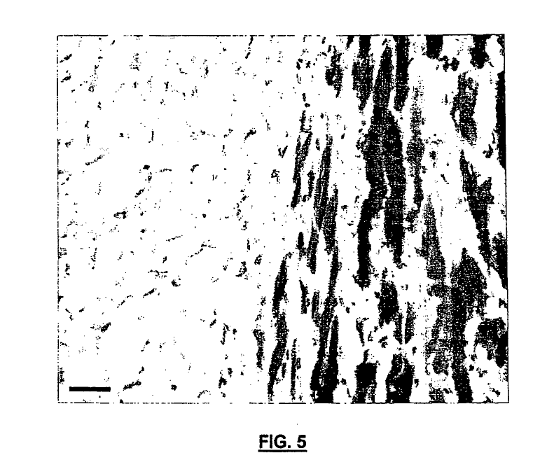

Upper left corner: immunohistochemical detection with the MY32 antibody in ewe skeletal muscle. The intensely labeled fibers (“fast twitch”) (in dark brown) and the weakly labeled fibers (“slow twitch”) characteristic of a muscle biopsy are noted (scale bar, 25 microns).

Upper right corner: immunohistochemical detection with the MY32 antibody in the ewe myocardium grafted with cells that have not undergone any culturing in vitro (scale bar, 25 microns).

Lower left corner: immunohistochemical negative control with the MY32 antibody in a normal ewe heart (scale bar, 250 microns).

Lower right corner: immunohistochemical negative control with the MY32 antibody in a ewe heart injected with medium alone (scale bar, 250 microns).

FIG. 5 illustrates the codetection of grafted skeletal muscle cells and of cardiomyocytes with the MY32 antibody and an antibody directed against connexin-43, a protein specific for gap junctions (red dots). This experiment did not demonstrate the presence of connexin-43 in the grafted cells or between the grafted cells and their microenvironment.

### DETAILED DESCRIPTION OF THE INVENTION

The present invention therefore relates to the development of a method for preparing animal or human stem cells which preserves the diversity and plasticity of these cells preferably originating from a tissue biopsy for their subsequent use in the context of tissue regeneration or repair by direct transplantation of the cells, without in vitro expansion in culture, thus prepared, in an animal or in humans.

Thus, it is understood that the initial preparation originating from a tissue biopsy is heterogeneous; for example, that originating from a skeletal muscle biopsy can contain myoblasts, fibroblasts, adipocytes, peripheral nerve cells, endothelial cells and smooth muscle cells, in addition to the pluripotent stem cells.

i) Definitions

The term “stem cell” is intended to mean cells which have both the ability of self renewal and the ability to differentiate into various types of cell precursors. The expression “diversity of the stem cells” is intended to mean the stem cells which are classified according to their developmental stage and to their tissue origin. More particularly, it is intended to mean a classification of embryonic or adult stem cells according to their tissue origin, for example skin stem cells or neuronal stem cells or muscle stem cells.

The expression “plasticity of the stem cells” is intended to mean the capacity for these cells to cross line borders, for example the ability of hematopoietic cells to differentiate into hepatocytes.

The term “tissue regeneration” is intended to mean the ability to reform a tissue either by activation of the progenitor cells of the tissue (for example skin tissue, liver tissue, heart tissue, bone tissue or nerve tissues) or by direct transplantation, without in vitro expansion in culture, of these progenitor cells. More particularly, it therefore involves tissue neoformation.

The term “tissue repair” is intended to mean an operation which makes it possible to overcome a deficiency without having to resort to a process of regeneration. Tissue repair results in an exogenous provision of cells which may be different from the cells of the recipient tissue.

The term “cell transplantation” is intended to mean an operation which is characterized by the provision of isolated cells. This transplantation may be carried out, for example, by injection directly, without in vitro expansion in culture, into the tissue or into the afferent circulation.

The term “animal” is intended to mean any living organism which may be subjected to cell transplantation, and this includes vertebrate beings such as human beings, domestic animals and wild animals, in particular birds.

ii) Method for Preparing Stem Cells

A first aspect of the present invention is directed toward a method for preparing human or animal stem cells. This method consists essentially of the following steps:

a) cell extraction;

b) mechanical dissociation;

c) enzymatic dissociation; and

maintaining of the cells in a specific medium for preserving the diversity and plasticity, said method excluding cell culture, i.e. an in vitro expansion phase in culture.

More precisely, the cell extraction is obtained first of all subsequent to a biopsy so as to provide a tissue fragment, such as a muscle, liver or skin fragment. For example, a muscle biopsy is taken under local anesthetic, subsequent to an incision, or else by needle. The tissue fragment thus obtained is then wetted in a defined medium, such as DME/202, in the presence of protective factors and of factors for inhibiting cell differentiation (Pinset and Montarras (1994), Cell Biology: A Laboratory Handbook, Academic Press).

Preferably, the defined medium is a medium hereinafter referred to as DPM (Diveristy Protecting Medium) and comprises at least:

a basic nutritive medium buffered with CO₂ concentration-dependent or -independent buffers. The media used consist, in most cases, of a mixture, in a 1:1 ratio, of medium of DME type and of F12 type. Among these, mention may be made, for example, of the DME/F12 and DME/MCDB 202 mixture;

a protective factor, such as:
antioxidants (ascorbic acid, N-acetylcysteine)
anticaspases (dose: approximately 0.1 mU to 10 mU)
metabolism protectors (L-carnitine)
metal protecting factors (transferrin) (dose: approximately 0.1 μg/ml to 100 μg/ml);

hormones such as glucocorticoids, insulin, retinoic acid, thyroid hormone, mineralocorticoids, IGF1 or IGF2, at physiological doses preferably ranging from 10⁻⁶ to 10⁻⁹ M;

differentiation inhibiting factors, such as:
FGFs (Fibroblast Growth Factors). Among the factors of the FGF family, three factors are particularly important and play a similar role, i.e. FGF-2, FGF-6 and FGF-10. Each of the factors of the FGF family is used at concentrations preferably ranging from 0.1 to 100 μg/ml;
EGF (Epidermal Growth Factor) (dose: approximately 0.1 μg/ml to 100 μg/ml);
LIF (Leukemia Inhibitory Factor) (dose: approximately 0.1 μg/ml to 100 μg/ml) [noncellular fraction of blood after clotting];
serum is also a factor that inhibits differentiation and can be used at a concentration ranging from 0 to 100%.

This DPM medium thus defined is used in the cell preparation method. The absence of animal fluid in the defined medium, such as serum for example, makes it possible to guarantee better infectious safety against viruses, prions, etc. and better reproducibility of the method of the invention. The samples originating from biopsies are maintained either at ambient temperature, or are stored, preferably, between 4 and 6° C. in the DPM for a period preferably not exceeding 48 hours. It is clear that the precise composition of the DPM may vary according to the type of tissue biopsy.

In step b) of the method, the tissue fragment maintained in the DPM medium is first thinly sliced until a cell homogenate is obtained. This is preferably done using sterile surgical scissors, but any other similar tool may be used. Subsequently, some of the erythrocytes and some of the adipocytes are preferably removed from the homogenous tissue extract or the tissue homogenate by means of a step consisting of washing and of centrifugation by gentle sedimentation at from 1 to 10 g (Pinset and Montarras. 1994. Cell Biology: A Laboratory Handbook. Academic Press).

In step c) of the method, the tissue homogenate obtained in step b) is subjected to an enzymatic digestion in order to optimize the cell dissociation. During this step of the method of the invention, proteolytic enzymes of bacterial origin, such as collagenase and/or pronase, are preferably used. However, for health safety reasons the use of trypsin produced by genetic engineering or of any other acceptable enzyme may be envisioned. These enzymes can be used alone or in combination at concentrations preferably ranging from 0.1 to 0.5%.

By way of nonlimiting example, the following procedure may be carried out during the implementation of step c) of the method:

the tissue fragment pellet obtained in step b) is suspended in DPM medium supplemented with collagenase at a final concentration of 0.4 g per 100 ml. The enzymatic digestion, ranging from 5 to 20 minutes, can be repeated several times. The temperature of the enzymatic reaction is preferably between 20° C. and 37° C. with external agitation. The enzymatic digestion is preferably carried out in several steps, with up to five steps. At each step, the fragments and the cells are separated by controlled centrifugation at 10 g. The fragment pellet is then resuspended in the DPM medium supplemented with proteolytic enzymes and then subjected to a further digestion step. The cells present in the supernatant are harvested by centrifugation at 200 g and resuspended in DPM medium. A cell suspension is thus obtained which combines the extracted cells of the various digestion steps. The cell suspension is then filtered through a nylon filter, the pore diameter of which is preferably approximately 34 microns, in order to remove the tissue fragments.

Thus, by means of the method for preparing stem cells according to the present invention, it is possible, for example, to prepare, safely and reproducibly, without in vitro expansion in culture, a cell suspension derived from a muscle biopsy which maintains its cell plasticity and colonization potential. For example, for one gram of tissue, it is possible to prepare from 1×10⁶ to 2×10⁶ cells. The effectiveness of the methods for maintaining the cell diversity and plasticity is evaluated by means of in vivo tests and ex vivo tests. In the first, the cell suspensions obtained are reintroduced into the animal after labeling. By way of example, a fluorescent molecule such as Green Fluorescent Protein will be used. The cell labeling makes it possible to follow the outcome of the cells injected into the organism, and to analyze their contribution to the various tissues and the differentiation of the cells injected. Under the conditions in which the diversity and plasticity are maintained, the injected cells contribute to the neoformation of various tissues, such as skeletal and cardiac muscle, vessels, tendons, cartilage, bone or hematopoietic tissue. This diversity and this cell plasticity are dependent on the injection site, underlining the importance of the environment in the cell' outcome.

The ex vivo tests (in culture) also make it possible to measure the cell diversity and the cell plasticity. These tests are based on clonal analysis and on the response to various differentiation inducers. Among these, mention may be made of:
growth factors such as insulin and IGFs, TGFs, BMPs, VEGF;
hormones such as retinoic acid derivatives, thyroid hormones, glucocorticoids;
extracellular matrix factors;
vitamins and agents for mineralization;
extracellular matrix components.

The cell identification is carried out by immunoanalyses using specific staining and specific antibodies, which allow both identification and quantification, and by transcriptome analysis.

The complementary in vivo and ex vivo approaches make it possible to measure the cell diversity and plasticity qualitatively and quantitatively and to thus validate the conditions for maintaining the cell diversity and the cell plasticity.

The cells thus prepared without in vitro expansion in culture are a source of appropriate cells for cell therapy and can be either reimplanted directly into an organism, or conserved for subsequent reimplantation.

The method of the present invention may comprise an additional step, i.e. a freezing step. This optional step of the method offers the following advantages:
conservation of the tissue samples;
dissociation between the preparation of the biopsy and the cell reimplantation;
biological and health-related characterization before cell reimplantation.

By way of nonlimiting example, the following procedure may be carried out during this freezing step:

10⁶ to 2×10⁶ cells originating from 1 gram of tissue are placed in DPM medium supplemented with cryopreserving agents, such as DMSO. The concentration of DMSO used is preferably 10%. Under these conditions, the cells are maintained at 20° C. for a period of 10 minutes and the temperature is then slowly, over a few hours, brought to minus 80° C. Finally, the cells are transferred into liquid nitrogen. It is important to note that these conservation conditions have the advantage of not modifying the cell characteristics.

Before carrying out the cell transplantation in the context of future clinical applications, it may be preferable to characterize the cell suspension obtained by the method according to the present invention. This characterization can be undertaken at the protein level by using cell markers such as:
P-Cam as a marker for endothelial cells;
N-Cam as a marker for neuronal and muscle cells;
smooth muscle actin as a marker for smooth muscle cells;
GFAP as a marker for glial cells;
Myf5, Pax3, Pax7, C-met and M-cadherin as markers for muscle cells;
Sca1+, C-Kit, CD45 and CD34 as markers for stem cells.

This characterization can also be undertaken at the transcriptional level by using biochips containing oligonucleotides encoding cellular genes (for example, specific transcription factors and factors of the cell cycle machinery) for identifying the cells of the cell suspension.

In accordance with the invention, said stem cells are animal or human stem cells chosen from the group consisting of progenitor stem cells for a tissue.

Also in accordance with the invention, the tissue is chosen from the group consisting of skin, liver, heart, bone and nerve tissues.

iii) Therapeutic Uses

A second aspect of the present invention is directed toward the use of a cell suspension or of stem cells obtained by means of the method of the invention, in therapeutic applications, including gene therapy, subsequent to transplantation of these cells in a human being, for example. More particularly, the present invention proposes to use the cells prepared by means of the method of the invention in the context of tissue repair and/or regeneration by cell transplantation without in vitro expansion in culture. The cells thus prepared will also be used as vectors in gene therapy.

For example, the adult stem cells obtained by means of the method of preparation according to the invention may be used in the context of bone tissue repair. In the present case, this involves transplanting the cells directly, without in vitro expansion in culture, onto a site of bone repair. In this particular microenvironment, the cells, or some of them, can adopt the bone phenotype and can thus contribute to the repair of the damaged tissue.

The cell preparation method of the present invention can also provide a suspension of mammalian adult stem cells which can be used in hematopoietic potential restoration. In fact, the ability of stem cells from a muscle biopsy to colonize the bone marrow of irradiated mice and to contribute to all the hematopoietic lines means that the repairing potential of muscle biopsy cells has to be considered in the case, for example, of leukemias.

Furthermore, it is known that cultured muscle cell grafts in muscle are relatively ineffective and are characterized by massive death of the reimplanted cells in the hours which follow their injection. It is, consequently, proposed that the method for preparing stem cells according to the invention, which does not comprise an in vitro expansion phase in culture, may contribute to muscle tissue repair. The cells contained in the suspension will be able to respond to the microenvironments and to thus restore functions or repair the muscle tissue.

Consequently, a subject of the invention is also the use of the human or animal stem cells obtained according to the method according to the invention, for preparing a medicinal product intended for the treatment of diseases by cell therapy or gene therapy.

A subject of the present invention is also a stem cell obtained according to the method according to the invention.

A subject of the present invention is also a method of treatment comprising a step consisting in implanting autologous or heterologous animal stem cells obtained according to the method according to the invention, in an animal or in humans.

A subject of the present invention is also a cell composition comprising human or animal stem cells obtained according to the method according to the invention.

According to an advantageous embodiment of said composition, the stem cells have the ability to colonize and the ability to allow functional recovery.

### EXAMPLE

The following example serves to illustrate the extent of the use of the present invention and not to limit its scope. Modifications and variations may be introduced therein without departing from the spirit or from the scope of the invention. Although it is possible to use other methods or products equivalent to those that are found below for testing or implementing the present invention, the preferred material and methods are described.

### Example 1

### Cardiac Tissue Repair

Introduction

Unlike skeletal muscle, cardiac tissue does not possess, in the adult state, stem cells capable of repairing this tissue after a lesion. As a result of this, cardiac ischemia is always accompanied by a contraction insufficiency which, if it is considerable, results in heart failure. The aim of cell therapy in this indication is to restore the contraction function. Experiments along these lines have been carried out in humans with muscle cells amplified in culture as a source of cells. This first pathway shows the feasibility of the approach, but also certain limitations of the adopted strategy. The amplification of a large number of muscle cells in media comprising animal fluids such as fetal calf serum, and their transplantation into heterotypic tissue, are operations that are both laborious and not without potential danger to health (viral contaminants, prions, etc.). The essential limitation of this method lies in the fact that the cells thus injected have a restricted plasticity which gives only skeletal muscle cells. The method for preparing stem cells of the present invention, without an in vitro expansion phase in culture, avoids depletion of the cell diversity through culturing.

Description of Cardiac Transplantation in Ewes

Anesthetic protocol: premedication with midazolam, induction using etomidate, tracheal intubation and ventilation at positive pressure with isoflurane in 100% oxygen. Monitoring is performed by electrocardioscopy, invasive arterial pressure, capnography. Postoperative analgesia with bupivacaine (intercostal block during thoracotomy), morphine and flunixine meglumine.

Skeletal muscle biopsy: A skeletal muscle biopsy (approximately 10 g) is sterilely explanted from the left femoral biceps of each ewe. The tissue thus removed is maintained in DMEM (SIGMA) or DPM, as defined above, at room temperature until mechanical or enzymatic digestion. The animal's wound is closed in the usual manner. The animals recover for approximately three hours, and are then re-anesthetized when the cells are ready to be reimplanted.

Muscle cell extraction: The skeletal muscle explants are weighed and then washed in DMEM or DPM. The adipose tissue and fascia are removed and the muscle is cut thinly with scissors until a tissue homogenate is obtained. The muscle fragments are then sedimented in DMEM or DPM at 300 rpm for 2 minutes and then the supernatant is removed.

In order to release the satellite cells, the muscle fragments are incubated at 37° C., with agitation, in 10 ml of DMEM or of DPM supplemented with 0.4% (W/V) of crude collagenase (type IA, SIGMA). After 20 minutes, the fragments are centrifuged at 300 rpm for 2 minutes.

The supernatant containing the isolated cells is conserved in DMEM containing 20% (V/V) of fetal calf serum, or DPM. The pellet undergoes up to four further digestions (Pinset, C. and Montarras, D. Cell Systems for Ex-vivo Studies of Myogenesis: A Protocol for the Isolation of Stable muscle cell populations from newborn to adult mice in Cell Biology: A Laboratory Handbook; second edition, e. J. F. Celis, Academic Press; 1998).

The extracted cells are then filtered through a nylon filter with pores having a diameter of 250 μm (Polylabo SA). The cells thus prepared do not undergo any in vitro expansion phase in culture.

Cell labeling: In order to label their nuclei, the cells are resuspended in 10 ml of serum-free DMEM containing 25 μg/ml of 4′,6-diamino-2-phenylindole (DAPI, SIGMA) for 10 minutes, or DPM under the same conditions. The cells are rinsed four times in DMEM or DPM in order to remove the DAPI. The mononuclear cells are counted with a counting cell under a fluorescence microscope. The cell preparation is then resuspended in 1.2 ml of serum-free DMEM or DPM and stored at 4° C. until implantation, without undergoing an in vitro expansion phase in culture.

Cell graft: The ewes, anesthetized beforehand according to the method described above, are placed lying down on their right side for a left thoracotomy on the 5th intercostal space. After pericardiotomy and suspension of the pericardium, the cells in DMEM or DPM are injected (0.1 ml per site) using an insulin syringe connected to a 27 G epijet, in 10 zones of the left ventricle. A mapping of the coronary vessels is established in order to pinpoint the injection sites in the myocardium. Epicardial pinpointing sutures are inserted for at least two injections. The thorax is closed in the usual manner and the animals recover under an analgesic system (morphine at 0.5 mg/kg IM BID, flunixine 1 mg/kg IM once) until the following day, inclusive.

Control cell cultures: In order to confirm the presence of muscle precursor cells in the cell preparation, a 100 μl sample of the cell suspension is seeded in a culture dish containing DMEM or DPM with fetal calf serum. The cells are maintained as a control in a 5% CO₂ atmosphere. Three to seven days after seeding, the cells are used for immunodetection of the specific regulatory factor MyoD (DAKO) as described by D. Montarras et al. (Cultured Myf5 NulI and MyoD NulI Muscle Precursor Cells Display Distinct Growth Defects. Biol Cell 2000; 92: 565-72).

Results

The cell injection procedure has no serious effect on the animals. A ventricular arythmia occurs during insertion of the needle and during injection of the cells, but is resorbed when the needle is withdrawn. Cell counting makes it possible to determine the injection of 1.7+/−0.3×10⁷ mononuclear cells labeled with DAPI.

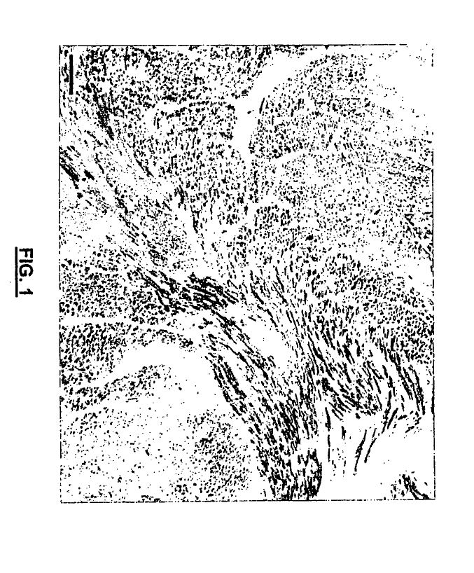

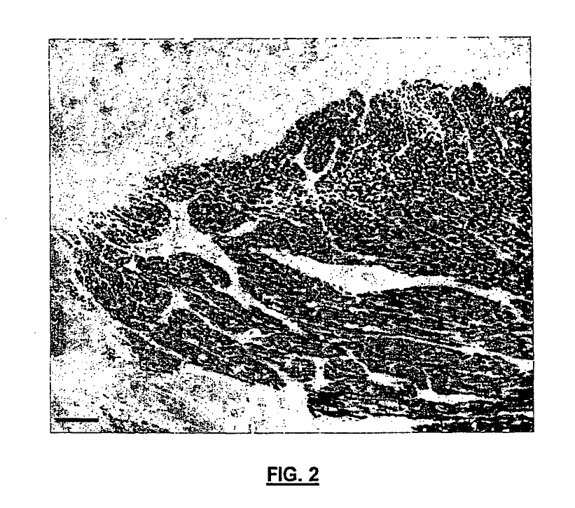

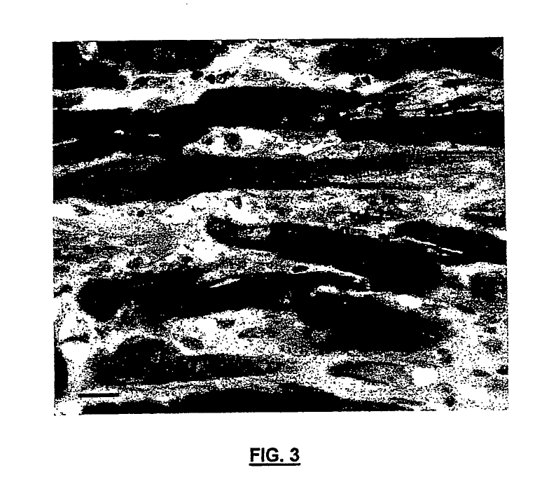

Expression of the heavy chain of skeletal myosin (MY32) is detected 3 weeks post-implantation in 8 animals out of 8, which confirms the cell survival and the myogenic expression of the implanted cells. Large surface areas of graft (from 2 to 9 mm in diameter) or discrete loci are observed inside the myocardial wall (FIG. 1). Isolated cells are also observed in the adipose tissue of the pericardium. Inside the myocardial wall, the fibers positive for MY32 expression are generally aligned with native cardiomyocytes. Some of these fibers are highly labeled with the antibody directed against MY32, whereas others are only weakly labeled (FIG. 2). These fibers are not electromechanically coupled to one another or to the native cardiomyocytes, as demonstrated by negative immunohistochemistry with connexin-43.

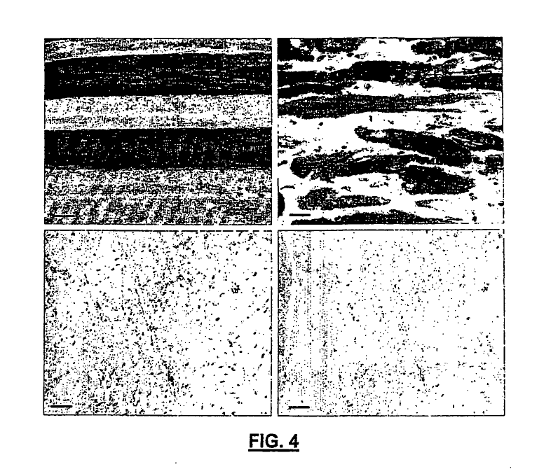

The implanted cells develop organized sarcomeres and exhibit either an elongated morphology characteristic of fused multinuclear myotubes, or a morphology which remains mononuclear (FIG. 3). These skeletal muscle cells have nuclei that are observed at the periphery or at the center. A replacement fibrosis and regions exhibiting mononuclear cells of the inflammatory response are also observed.

No labeling with DAPI is observed in the explanted hearts. This may be due to active cell division resulting in dilution of the DAPI. In fact, in the cell cultures acting as controls, the DAPI becomes undetectable after 6 to 8 doublings of the cell population.

The culture dishes acting as controls are observed daily. Immunodetection of the skeletal muscle-specific regulatory factor MyoD shows that approximately 50% of the cells in culture are skeletal muscle precursor cells. When the cells are allowed to fuse, all the cells show numerous myotubes one week after seeding.

Although preferred embodiments of the invention have been described in detail above and illustrated in the attached drawings, the invention is not limited to only these embodiments, and several changes and modifications may be introduced therein by those skilled in the art without departing from the context or from the spirit of the invention.

1. A method for preparing human or animal stem cells, said method comprising:
cell extraction;
mechanical dissociation;
enzymatic dissociation;
maintaining of the cells obtained, in a specific culture medium for preserving the diversity and plasticity,
said method excluding cell culture.

2. The method as claimed in claim 1, wherein said specific culture medium comprises at least:
a) a nutritive medium;
b) a protective factor;
c) hormones;
d) differentiation inhibiting factors.

3. The method for preparing stem cells as claimed in claim 1, wherein said stem cells are animal or human stem cells selected from the group consisting of progenitor stem cells for a tissue.

4. The method as claimed in claim 3, wherein the tissue is selected from the group consisting of skin, liver, heart, bone and nerve tissues.

5. A medicinal product comprising the human or animal stem cells, obtained according to the method of claim 1,. and one or more additives.

6. A stem cell obtained according to the method of claim 1.

7. A method of treatment comprising implanting autologous or heterologous animal stem cells, obtained according to the method of claim 1, into in an animal in need thereof.

8. A cell composition comprising human or animal stem cells, obtained according to the method of claim 1.

9. The composition as claimed in claim 8, wherein the stem cells have the ability to colonize and the ability to allow functional recovery.

10. A method of treating disease by cell therapy or gene therapy, comprising, injecting the human or animal stem cells, obtained by the method of claim 1, into a subject in need thereof.

11. The method for preparing stem cells as claimed in claim 2, wherein said stem cells are animal or human stem cells selected from the group consisting of progenitor stem cells for a tissue.

12. The method as claimed in claim 11, wherein the tissue is selected from the group consisting of skin, liver, heart, bone and nerve tissues.
